(19) United States
(12) Patent Application Publication
(10) Pub. No.: US 2025/0107560 A1
(43) Pub. Date: Apr. 3, 2025

(54) CHRONOBIOLOGICAL FOOD FOR INFANTS AND METHOD FOR PRODUCING SAME

(71) Applicant: Ekkehart PADITZ, Dresden (DE)

(72) Inventor: Ekkehart PADITZ, Dresden (DE)

(21) Appl. No.: 18/728,838

(22) PCT Filed: Jan. 12, 2023
(86) PCT No.: PCT/EP2023/050674

§ 371 Date: Jul. 12, 2024

(30) Foreign Application Priority Data
Jan. 13, 2022 (WO) PCT/EP2022/050676

(51) Int. Cl. A23L 33/00; A23L 33/10; A23L 33/175

(52) U.S. Cl. CPC A23L 33/40; A23L 33/10; A23L 33/175

(57) ABSTRACT

A method for producing infant formula, in particular infant starter formula, is described. The method is characterized in that non-pooled milk of a mammal is provided as starting material for the production of the infant formula. Further addressed are an infant formula as such, the use of non-pooled milk, in particular as chrononutrition, and melatonin and melatonin-containing infant formula for use in a method for the prevention and/or treatment of sleep disorders in infants and/or in a method for the prevention of conditions and their consequences which may arise due to melatonin deficiency in the first months of life.

The present invention relates to an infant formula and a method of its preparation. The invention further addresses the use of non-pooled milk for the preparation of infant formula and the use of melatonin and melatonin-containing infant formula for use in a method for the prevention and treatment of sleep disorders in infants and other disorders which may be associated with melatonin deficiency.

The objective of the present invention is to provide an improved infant formula. A further objective is to enable the infant to sleep well and to support it in the formation of the day-night rhythm. A further objective is to influence conditions and their consequences that may be associated with melatonin deficiency in the first months of life.

When a baby is born, its natural first food is breast milk, which is optimally adapted to the baby's needs. For this reason, the World Health Organization (WHO) recommends that babies should be exclusively breastfed for the first six months. Representative studies show that in Germany, for example, the initial breastfeeding rate has increased continuously since the 1990s and has risen to over 80% since the 2003/2004 birth cohorts. However, the breastfeeding rates for exclusive breastfeeding for at least four and six months were only 40.0% and 12.5% respectively for the 2012 to 2016 birth cohorts in Germany. Since complementary foods cannot be given up to and including the 4th month of life, 60% of all infants receive an industrial alternative to breast milk before complementary foods are introduced. According to a study that investigated breastfeeding behavior in Switzerland in 2014, as many as 95% of mothers had breastfed their child from the start. Over 50% of children were exclusively breastfed for at least 12 weeks of their lives, with around 60% still being fully breastfed up to and including the 4th month of life. The remaining 40% in turn rely partially or completely on an industrial alternative to breast milk.

For all those infants who—for whatever reason—cannot be breastfed, there is therefore an alternative in the form of industrially produced ready-made infant formula, which is divided into infant starter formula, follow-on formula and complementary food. Infant starter formula is specifically intended for feeding in the first six months of life and is the focus of the present invention. With a history dating back to the middle of the 19th century, ready-made infant formula has changed massively over the course of time and the infant starter formula especially has become ever closer to the original, breast milk. Nevertheless, breast milk is still a big step ahead of modern infant starter formula. For example, breast milk not only nourishes babies, but also protects them from infections. This is due to the antibodies and complex sugar molecules contained in the milk, which help to colonize the intestines with good bacteria.

Although industrial infant starter formulas contain many nutrients, they do not offer anywhere near the same level of immune protection. The composition of breast milk also changes constantly, not just over the course of weeks and months, but even within minutes: at the beginning of a feeding period, breast milk is more watery to quench the infant's thirst; towards the end, the fat content increases significantly to fill the infant up. In addition to the change in composition during the first months of life and during a breastfeed, the composition of breast milk also changes during the day. All in all, it can be said that breastfeeding is far superior to feeding industrial infant formula, which shows no variation within a product and lacks many of the unique characteristics in terms of nutrients, hormones and immune defense.

One of the objectives of the present invention is to produce an improved industrial infant formula which, in particular, better reflects the chronobiological change in the composition of breast milk over the course of a day.

This objective is solved by a process for producing infant formula, in particular infant starter formula, according to claim 1.

This refers to a process for the production of infant formula, which is characterized in that the milk provided as starting material for the production is non-pooled milk. This comes from at least one mammal, the mammal or mammals preferably being cloven-hoofed animals, in particular cows, sheep, horses, donkeys and/or goats. Due to the rather sensitive stomachs of infants, the use of milk from only one mammal species—in particular cow's milk—is preferred in accordance with the recommendations of the professional societies. Nevertheless, a combination of two or even more milks from different mammals is possible. The term “infant” stands—in accordance with general professional knowledge—for a child in the first year of life, i.e. a baby from 0 to 12 months, whereby the term “child” in turn refers to a human in the childhood stage of life.

“Animal children” therefore do not fall under the term “infant”. Even if, biologically speaking, the human counts to the mammals, it is usual in the technical field of the present invention to distinguish between the milk of a mammal and breast milk (human milk). Consequently, the skilled person does not consider breast milk to be the milk of a mammal. When the invention refers to the milk of mammals, only the milk of animals suckling their young is meant and not human breast milk.

In other words, the method comprises the step of providing non-pooled milk from one or more mammals, preferably from one animal species. In most cases, industrial infant formula is a milk powder enriched with various fats, trace elements, vitamins, sodium, magnesium and calcium, among other things. Accordingly, the process may comprise the step of adding fat(s), trace elements, vitamins, sodium, magnesium and/or calcium. If the infant formula is to be provided in powder form, the free water is removed from the non-pooled milk, for example by heating and/or with negative pressure, in order to convert it into milk powder. Between six and seven liters of non-pooled fresh milk are required to obtain one kilogram of dry milk powder.

Typically, powdered infant formula is produced as described below. After the raw milk has been provided and treated, a liquid mixture is made from it and various powdered additives, such as for example lactose and protein. A high-shear mixer ensures that the additives dissolve in the milk. A large proportion of the water contained in the milk enriched with additives is removed by evaporation and dried in a spray dryer to form a free flowing, well soluble, agglomerated powder.

Before the resulting spray-dried powder is then portioned and filled, it is mixed with further additives, in particular temperature-sensitive additives, such as for example minerals and vitamins.

In principle, pooled milk is a milk that is made up of milk units or milks from at least two milking processes that in particular have taken place at different times spread over the course of a day. A milk unit means a certain volume or a certain quantity of milk, which in particular originates from one milking process. A milking process can be understood as the milking of a single mammal as well as the milking of all animals or only some of the animals, whereby the milking process is characterized by the fact that it takes place within a limited time frame. Cows usually enter the milking parlor twice a day. There are two milking processes, meaning that each animal—and therefore all the animals—is milked twice a day, usually once in the morning, e.g. at 7.30 o'clock, and once in the evening, e.g. at 18.00 o'clock. The average time interval between the milk unit from the morning milking process and the milk unit from the evening milking process is therefore 10.30 hours. With 30-40 cows and one worker, a milking process takes about 1 hour. Non-pooled milk in the sense of the invention is therefore the milk of a milk unit which originates from only one milking process and was obtained in particular at a specific time of day, or milk from several milk units, whereby the milk units can originate from several milking processes which were however obtained in a narrow time window seen over 24 hours (e.g. the narrow time window is at least less than 12 h, preferably less than 9 h, further preferably less than 6 h or even only 3 h, 2 h or 1 h, e.g. from 17:00 to 18:00 o'clock). However, the 24 hours need not represent the same day, i.e. milk that is composed of a first milk unit obtained on day X at a specific time of day and inert of a narrow time window, and a second milk unit obtained on day X+n (n=natural number) at the essentially identical specific time of day and inert of the essentially identical narrow time window, also qualify as non-pooled milk. The time windows are preferably selected in such a way that the composition of the milk essentially does not change during this time with regard to at least one target parameter (e.g. melatonin), which is subject to chronobiological changes in the course of a day. The pooled and non-pooled milk can generally come from one barn or one farmer, but also from several barns or farmers. Relevant for distinction between pooled and non-pooled milk is essentially only the temporal regime of the milk obtaining seen over a 24-hour cycle. This is due to the chronobiological changes in the composition of the milk as described below.

Non-pooled milk within the meaning of the invention is further characterized by the fact that chronobiological changes within a period of 24 hours are retained in the composition of the milk and are not neutralized by mixing with milk from a milking process with an essentially different time of day (e.g. a milking process outside the narrow time window). Accordingly, milk from a milking process that is 24 hours apart from the previous milking process is non-pooled milk within the meaning of the invention, since chronobiological changes in the composition of the milk over the course of the day would still be recognizable.

The target is to use non-pooled milk to produce milk powder for infant formula that does not have an average melatonin concentration, but either a high melatonin concentration or a low melatonin concentration. Melatonin is a hormone that is synthesized from serotonin in the human organism and in mammals and has a significant influence on the day-night rhythm of the human body. Melatonin is also a metabolite of the tryptophan metabolism, the production of which is controlled by the daily rhythm and inhibited by light. It has a biological half-life of 30-45 minutes, is metabolized in the liver and approx. 95% of it is excreted in the urine as 6-sulfatoxymelatonin.

The concentration of melatonin in breast milk changes over the course of a day. This is particularly important as infants have a reduced concentration of melatonin in their blood from the first days of life until the 2nd, 3rd to 4th month. In psychomotor retarded babies, this is even the case up to the 6th month. Prenatally and in the first 8 weeks of life, no melatonin synthesis takes place in the pineal gland; a maximum melatonin concentration is not even measured until the age of 3-7 years. There is evidence that the pineal gland can react effectively and very quickly in the darkness by synthesizing and releasing melatonin from the 3rd month of life at the earliest. Melatonin from breast milk therefore ensures healthy infant development and a good sleep. Furthermore, melatonin from breast milk controls the infant's day-night rhythm, which the infant only begins to develop itself in the 2nd to 3rd month of life. Melatonin therefore plays an important role in harmonizing the infant's rhythm with the mother's rhythm. The fact that breastfed infants cry less, sleep more peacefully and are less often considered to be crybabies is also linked to the better chronobiologically adjusted melatonin supply of breastfed infants. Since breast milk and cow's milk (and also the milk of other mammalian species such as e.g. goat's milk) are subject to comparable day-night fluctuations with regard to the melatonin concentration, the exclusive or at least predominant use of cow's milk as the starting material is preferred in one embodiment.

It was found that the addition of the melatonin precursor tryptophan to infant formula can have positive effects on sleep parameters measured by actigraphy and on parental satisfaction in infants with sleep disorders. However, the acrophase of tryptophan is several hours ahead of the acrophase of melatonin, so that the administration of tryptophan should be well timed. The acrophase is the time span between zero o'clock and the maximum value for a circadian rhythmically varying parameter. This is often specified together with the nadir, the range of the corresponding minimum value. Among other things, this makes it difficult to control the melatonin concentration in the blood by adding tryptophan. Although tryptophan, or more precisely L-tryptophan, is already added to industrial infant formula, this is in a concentration that is too low to have a positive effect on the infant's sleep and health. As industrial infant formula makes no distinction between day and night, this is not surprising, as it is not desirable for the infant to sleep all day and thus not be able to develop a day-night rhythm at all. In addition, the complexity of the relationships between melatonin, insulin (melatonin as an insulin antagonist), long-chain neutral amino acids and other ingredients in breast milk prohibits a linear oriented intervention that would only consist of adding an ingredient such as melatonin or tryptophan to the infant formula of non-breastfed infants. For example, the long-chain neutral amino acids compete with tryptophan when it comes to crossing the blood-brain barrier. Insulin, in turn, contributes to the transfer of long-chain neutral amino acids into the muscle and thus increases the amount of tryptophan available for melatonin synthesis in the brain. The pure addition of tryptophan to infant formula is also not expedient because tryptophan is the only amino acid that is very tightly bound to the carrier protein album in the serum and because the membrane passage of tryptophan to cross the blood-brain barrier is inhibited by long-chain neutral amino acids. The availability of serotonin in the brain as an intermediate product in the synthesis of tryptophan to melatonin is therefore dependent on the quotient between tryptophan and long-chain neutral amino acids. It is not the tryptophan concentration, but the aforementioned quotient that has an influence on sleep parameters in infants and amounts to approx. 1:8 in breast milk. Consequently, in the industrial production of baby food, which should be adapted to breast milk, not only the content of tryptophan, serotonin and melatonin, but also the concentration of long-chain neutral amino acids (LNAA, Large Neutral Aminoacids) should be taken into account. In addition to tryptophan, the nine LNAAs include phenylalanine, tyrosine, histidine, methionine, threonine, leucine, isoleucine and valine. But even if this complex challenge were to be met, the melatonin intake would still not be sufficient because, on the one hand, although tryptophan is generally an important substrate in the metabolism, only 1 to 3% of tryptophan metabolism is used for the synthesis of serotonin and melatonin. On the other hand, the bigger problem is that tryptophan cannot be utilized for melatonin synthesis in the pineal gland until the 3rd month of life, as the sympathetic innervation of the pineal gland has not yet matured. This hurdle can be circumvented by using milk from a mammal as a starting product, which is collected in the morning or evening and administered in the morning as a stimulant and in the evening as sleep-supporting chrononutrition.

By directly targeting the chronobiological change in melatonin concentration in cow's milk through the use of non-pooled milk, the method according to the invention circumvents the attempt to mimic this complexity and provides an infant formula whose other components are naturally tuned to the melatonin concentration. In addition to melatonin, there are other ingredients whose concentration is subject to circadian fluctuations, such as fats, triglycerides, cholesterol, iron, cortisol and cortisone. These components can also be supplied to the non-breastfed infant in a more needs-oriented manner by using non-pooled milk as the starting material.

At this point it is noted that the invention in its entirety can be applied to infant formula in general, but is especially suitable for infant formula in particular. Infant formulae are characterized by the fact that they are the only foods apart from breast milk that fully meet the nutritional needs of infants during the first 6 months and completely cover their needs until the introduction of complementary foods (with the exception of vitamins D and K, which are nowadays given as supplements as standard). In general, infant formulae (usually referred to as “pre” or “1”) are best suited to breast milk and should be the first substitute for breast milk, regardless of when they are introduced. “Pre” milks contain lactose as the only source of carbohydrate (apart from any traces of glucose), while “1” milks can contain both lactose alone and small amounts of starch or other carbohydrates (usually maltodextrins, rarely sucrose). The whey-casein ratio for both is around 60:40. As breast milk has a very high lactose content and lactose has a prebiotic effect, lactose should be preferred as a carbohydrate source in infant starter formulae (source: https://www.pharmasuisse.org/data/docs/de/10841/pharManuel-Artikel-S%C3%A4uglingsern%C3%A4hrung.pdf?v=1.2; “Lebensphasen: Säuglingsernährung” by Jeannette Dommer Schwaller). The “Verordnung der Bundesministerin für Gesundheit, Familie und Jugend über Säuglingsanfangsnahrung und Folgenahrung” of the Austrian Federation (source: https://www.ris.bka.gv.at/eli/bgbl/II/2008/68), which bears the short title “Säuglingsanfangsnahrung und Folgenahrung” and was promulgated on Feb. 20, 2008, defines infant starter formula even more specifically on the basis of its ingredients. Infant starter formula is made from defined protein sources and other ingredients whose suitability for the special nutrition of infants from birth has been proven by generally recognized scientific findings. For example, the energy content is between 60 and 70 kcal/100 mL (for prepared food), the protein content is 1.8 to 3 g/100 kcal (for infant starter formula based on mammalian milk, in particular cow's milk), the max. taurine content is 12 mg/100 kcal, the choline content between 7-50 mg/100 kcal, the lipid content at 4.4 to 6.0 g/100 kcal and the carbohydrate content between 9-14 g/100 kcal, whereby only lactose, maltose, sucrose, glucose, malto-dextrins, (dried) glucose syrup, boiled or gelatinized starch may be used. Further limits can be found in the Federal Law Gazette “Säuglingsanfangsnahrung und Folgenahrung” and its annexes, which can be included in this application if required. For the sake of clarity, no limit values for ingredients for follow-on milk are given here, as these can also be taken from the Federal Law Gazette already cited and be included in this application.

In one embodiment of the method, which may be combined with any of the embodiments mentioned and to be mentioned, unless in contradiction, at least 50%ᵥₒₗ of the milk used as starting material is non-pooled milk. In other embodiments, at least 65, 70, 75, 80, 85, 90, 95, 98 or 99%ᵥₒₗ of the milk provided as starting material is non-pooled milk.

In general, the higher the proportion of non-pooled milk in the “starting material milk”, the more the melatonin concentration and the concentration of other chronobiologically changing substances in the produced industrial infant formula, and thus also in the ready-to-drink product, reflect the natural chronobiological change.

In one embodiment of the method, which may be combined with any of the embodiments mentioned and to be mentioned, unless in contradiction, essentially only non-pooled milk is used as starting material. Pooled milk is then, except perhaps in traces, not used as a starting material for the production of industrial infant formula.

If pooled milk is not used at all as a raw material for the production of industrial infant formula, the natural chronobiological change is maximally reflected.

In one embodiment of the method, which may be combined with any of the embodiments yet to be mentioned, unless in contradiction, the milk provided as starting material is obtained by milking between 3 o'clock at night and 7 o'clock in the morning. In particular, milking is carried out between 4 o'clock at night and 6 o'clock in the morning, preferably at 5 o'clock at night±15 min (i.e. between 4.45 o'clock and 5.15 o'clock).

The aforementioned inhibition of melatonin production is suspended in darkness in the biological night, so that the melatonin concentration increases during the night with a maximum concentration in humans at around 3 o'clock at night. In cows, for example, the melatonin concentration changes over the course of the day in a similar way to humans:

In the early evening, the melatonin concentration in their blood—and thus also in their milk with a slight time delay-begins to rise; by early morning, the melatonin concentration has returned to the low level of the rest of the day. Depending on the time of year and the associated shifts in the times of sunrise and sunset, the fluctuation throughout the whole day in cows is between approx. 5-8 pg/mL and approx. 35 pg/mL, with the maximum in the blood being reached between 20.00 and 23.00 o'clock. In order to obtain milk that is especially rich in melatonin, milking should therefore be carried out early in the morning, with milking in the early evening each time before (e.g. 8 to 12 hours before, preferably 9 to 10 hours before), so that the melatonin-poor milk of the day does not lower the concentration of the melatonin-rich “night milk”.

The melatonin concentration in cow's milk, for example, results from the interplay between a) melatonin synthesis (=in the pineal gland, in the retina and in the intestine), b) melatonin secretion into the mammary glands and c) melatonin storage in the cysters of the mammary glands. After sunset, the concentration of melatonin increases in the blood and, with a slight delay, also in the milk. This process stops after sunrise. As already indicated, the melatonin concentration does not only depend on the time of day: In the morning hours, blue light components predominate in natural light, which have a particular effect on the suppression of the melatonin system.

Exposure to light in the morning with corresponding blue light components therefore helps to inhibit the formation and secretion of melatonin during the day, so that melatonin activity can be induced all the more strongly in the evening. The effectiveness of melatonin as a sleep-inducing hormone is therefore not only dependent on its synthesis, storage and secretion in the evening, but is also determined by light exposure in the morning. Exposure to light in the evening can also contribute to a reduction in natural melatonin activity in the evening, which can lead to sleep disorders. Consequently, it is beneficial if the mammals whose milk is used as raw material are not kept in a darkened stable during the day, but are exposed to as much natural light as possible from sunrise to sunset. Nocturnal background lighting in the stable should not have a high blue light component.

In one embodiment of the method, which may be combined with any of the embodiments mentioned and to be mentioned, unless in contradiction, the milk provided as starting material has a melatonin content of from 10 pg/mL to 60 pg/mL inclusive. In particular, the milk has a melatonin content of from 20 pg/mL to 50 pg/mL inclusive and preferably from 20 pg/mL to 40 pg/mL inclusive. In other words, the milk provided as starting material preferably has a concentration of melatonin of at least 20 pg/mL, in particular of at least 30 pg/mL, and further in particular of at least 40 pg/mL.

Since breast milk has such a low melatonin concentration during the day that it is even below the detection limit or amounts only a few pg/mL, the concentration doubles towards the evening and at night in mature breast milk maximum values in the range of 20-30 pg/mL or even 40-50 pg/mL are present, a non-pooled milk from mammals with at least 10 pg/mL or better still with at least 15 or even 20 pg/mL is a good starting material for a night milk. Milk with a concentration of 20±10 pg/mL or even 40±10 pg/mL is particularly suitable for the production of infant starter formula. If one considers the total amount of melatonin produced by a mammal during the day, at least 70%, in particular at least 80%, further in particular at least 90%, or even at least 95% of this daily amount is found in the milk provided as starting material with melatonin concentrations as just described.

For example, a melatonin concentrations in blood plasma between 0.10 and 1.0 nM is optimal for triggering the chronobiological effects of melatonin.

In one embodiment of the method, which may be combined with any of the embodiments mentioned and to be mentioned, unless in contradiction, the milk provided as starting material is obtained by milking between 14:30 o'clock and 18:30 o'clock. In particular, milking is performed between 15:30 o'clock and 17:30 o'clock, preferably at 16:30 o'clock±15 min.

Since the melatonin concentration in the milk decreases sharply towards the morning and only increases again in the evening, milk with an especially low melatonin concentration can be obtained at the above-mentioned times, which can then be used for the production of infant starter formula that is specifically intended for feeding or nourishing the infant during the day.

In one embodiment of the method, which can be combined with any of the embodiments mentioned and to be mentioned, unless in contradiction, the milk used as starting material has a melatonin content of less than 10 pg/mL, in particular less than 8 pg/mL. The concentration of melatonin is further in particular below 5 pg/mL, preferably in the range from including 0 pg/mL up to and including 3 pg/mL.

As already mentioned, breast milk has such a low melatonin concentration during the day that it is even below the detection limit or amounts only a few pg/mL. If a non-pooled milk from a mammal is used as the starting material for infant formula, which has a very low melatonin concentration, this infant formula is perfectly suited to be fed during the day. If one considers the total amount of melatonin produced by a mammal during the day, a maximum of 30%, in particular a maximum of 20%, further in particular a maximum of 10%, or only a maximum of 5% of this daily amount is found in the milk provided as starting material with melatonin concentrations as just described.

In one embodiment of the method, which may be combined with any of the embodiments mentioned and yet to be mentioned, unless in contradiction, the non-pooled milk is obtained by a milking process carried out between 6 hours and 18 hours after the previous milking process. In particular, the non-pooled milk is obtained by a milking process carried out between 9 hours and 15 hours or preferably even between 11 hours and 13 hours after the previous milking process.

By a sufficiently long interval between the milking processes it is ensured that the melatonin-rich night milk is not mixed with the melatonin-poor day milk in the milk containers and that the desired melatonin concentrations are achieved in the milk obtained in each milking process. Preferably, only one milking process is carried out per night, as more frequent night milking can lead to a reduction in the melatonin concentration.

In one embodiment of the method, which can be combined with any of the embodiments mentioned and to be mentioned, unless in contradiction, the milk used as starting material is obtained by a milking process carried out shortly before or at sunrise.

At sunrise, melatonin production is inhibited, so that a milking process carried out at the beginning or onset of this inhibition ensures an especially melatonin-rich milk, in particular if the previous milking process took place when the inhibition was suspended, i.e. at sunset. To ensure that milk with a low melatonin content is not already being produced, milking can also be carried out shortly before sunrise, whereby shortly before means approximately one hour, in particular half an hour, before.

In one embodiment of the method, which may be combined with any of the embodiments mentioned and to be mentioned, unless in contradiction, the milk used as starting material is obtained by a milking process carried out shortly before or at sunset.

At sunset, the inhibition of melatonin production is suspended, so that a milking process at the end or with the suspension of this inhibition ensures an especially melatonin-poor milk, in particular if the previous milking process took place when the inhibition started, i.e. at sunrise. To ensure that milk with an increased melatonin content is not already produced, milking can also take place shortly before sunset, whereby shortly before means about an hour, in particular half an hour before.

In one embodiment of the method, which may be combined with any of the embodiments mentioned and yet to be mentioned, unless in contradiction, the method is a two-part method. In the first part, melatonin-rich milk is obtained as a first starting material by milking at a first time or in a first time window. In the second part, on the other hand, melatonin-poor milk is obtained as a second starting material by milking at a second time or in a second time window. The first time or the first time window is between 3 o'clock at night and 7 o'clock in the morning, in particular between 4 o'clock at night and 6 o'clock in the morning, preferably at 5 o'clock at night±15 min, or at sunrise. The second time or the second time window is between 14:30 o'clock and 18:30 o'clock, in particular between 15:30 o'clock and 17:30 o'clock, preferably at 16:30 o'clock±15 min, or at sunset. The interval between the first and the second part of the process is between 6 hours and 18 hours, in particular between 9 hours and 15 hours, preferably between 11 hours and 13 hours. The first starting material and the second starting material are processed separately, namely into a first melatonin-rich infant formula and a second melatonin-poor infant formula.

On the one hand, said method enables the production of a melatonin-poor infant formula, e.g. as a chrononutrition to wake up, and on the other hand, the production of a melatonin-rich infant formula, e.g. as an evening sleep-supporting chrononutrition. The method can of course be carried out several times in succession over several days, weeks, months and even years. Also, the first part does not necessarily have to be carried out first and then the second part, but the second part can also be carried out first on one day and the first part on the following day. Ideally, the milking of the first part is carried out at or before sunrise, which is between 5:30 and 8:30 o'clock depending on the time of year. The milking of the second part ideally takes place at or before sunset, which is between 16:30 and 21:30 o'clock depending on the time of year. The length of the day is accordingly between 8 and 16 hours, so that the time between the first milking in the morning and the second milking in the evening is also 8 to 16 hours, or the time between milking in the evening and milking in the morning is also 8 to 16 hours. The information applies to Zurich, Switzerland and may have to be adjusted for other locations according to the respective geographical time zone.

In one embodiment of the method, which can be combined with any of the embodiments mentioned and to be mentioned, unless in contradiction, the L-tryptophan concentration of the starting material, or of the first starting material in the two-part method, is adjusted. Preferably, the adjustment is carried out in such a way that the infant formula in the ready-to-drink state has a concentration of L-tryptophan of at least 15 mg/100 kcal, or even of at least 25 mg/100 kcal, such as for example from 25 to 37 mg/100 kcal, in particular from 30 to 34 mg/100 kcal, further in particular from 32 mg/100 kcal.

As already mentioned, tryptophan is an important starting material for a wide range of metabolic processes, of which only 1 to 3% is used for melatonin synthesis. The development of the body and brain is dependent on tryptophan and can absorb this essential amino acid and at least process it in the metabolism. However, tryptophan cannot yet be utilized for melatonin synthesis in the pineal gland until the 3rd month of life, as the sympathetic innervation of the pineal gland has not yet matured. It is therefore important that the infant formula fed in the evening and at night is made from non-pooled milk and therefore has an increased concentration of melatonin.

Healthy cows have a tryptophan concentration of approx. 7.2 to 7.3 μM in their milk. With a molar mass of 204.23 g/mol, this corresponds to a quantity of 1.47 to 1.50 mg/L. With an energy content of, for example, 66 kcal per 100 mL of ready-to-drink infant formula, i.e. approx. 152 mL per 100 kcal, cow's milk would only have 0.228 mg tryptophan in 152 mL, so that an addition of L-tryptophan as an important substrate in the metabolism makes sense.

In one embodiment of the method, which can be combined with any of the embodiments mentioned and yet to be mentioned, unless in contradiction, the quotient of L-tryptophan to long-chain neutral amino acids (LNAA) is adjusted. Preferably, the adjustment is carried out in such a way that the quotient is 0.126 to 0.134, in particular 0.128 to 0.132, further in particular 0.130.

Since tryptophan competes with the LNAAs for the transporter in the blood-brain barrier, a deliberately chosen quotient can ensure that a sufficient amount of tryptophan actually arrives in the brain for metabolization. L-tryptophan is always added because, unlike D-tryptophan, it is non-toxic. It also binds 100 times better to the carrier albumin.

In one embodiment of the method, which can be combined with any of the embodiments mentioned and yet to be mentioned, unless in contradiction, the amount of alpha-lact albumin is adjusted. Preferably, the adjustment is such that alpha-lact-albumin makes up 30 to 42%, in particular 33 to 39%, further in particular 36%, of the whey protein in the infant starter formula. Alternatively, alpha-lact-albumin is added in a maximum amount of 8.3 g/L relative to the ready-to-drink product.

Alpha-lact-albumin serves as a carrier for the L-tryptophan and thus ensures that it is available for further metabolization.

In one embodiment of the method, which can be combined with any of the embodiments mentioned and yet to be mentioned, unless in contradiction, the one mammal or the several mammals are exposed to a bright light of at least 200 lux during the day for at least 12 hours when the animals are kept in the stable, in particular for at least 14 hours, further in particular for at least 16 hours. The bright light preferably has a high proportion of blue light and has a wavelength of 465 nm, for example. Furthermore, the one mammal or the several mammals are exposed to darkness during the night for at least 8 h, in particular for at least 10 h, further in particular for at least 12 h, when the animals are kept in the stable.

In order to achieve the desired high melatonin concentration in the infant formula provided to the infant as night milk, also when using non-pooled infant animal milk from stable animals, the lighting situation in the stable can be optimized by shining bright light on the mammals preferably around 16 h in order to significantly suppress the activity of the melatonin system, so that they can react all the more strongly to the darkness in the evening/night—with increased melatonin secretion.

In one embodiment of the method, which may be combined with any of the embodiments mentioned and yet to be mentioned, unless in contradiction, the non-pooled milk is not heated above 60° C. during the method. Additionally or alternatively, the non-pooled milk is exposed to a pH of at most 7.4 throughout the method.

By not heating the non-pooled milk or the intermediate products based on the non-pooled milk to a temperature above 65° C., in particular above 62° C., further in particular above 60° C., in any method step on the way to the infant (starter) formula, the original melatonin content is largely retained and the melatonin is not destroyed. Temperature ranges for heating from inclusive 60° C. to inclusive 65° C., in particular from inclusive 60° C. to inclusive 62° C. are particularly preferred. The particularly critical steps here are those of treating the raw milk and removing the water. Comparable applies to a pH value regime of maximum 7.4 during the individual method steps, which also ensures that the amount of melatonin remains largely stable and that no destruction of the melatonin from the non-pooled milk can take place due to a too high pH value.

In one embodiment of the method, which can be combined with any of the embodiments mentioned and yet to be mentioned, unless in contradiction, at least one to all of the substances from the group of L-tryptophan, 5-HTP, serotonin, acetyl-serotonin, melatonin, long-chain neutral amino acids (LNAA) and alpha-lact-albumin originate from the starting material. This means that of the seven substances mentioned, one, two, three, four, five, six or seven of the substances get into the infant formula (exclusively) via the starting material. In particular, no other addition of one, two, three, four, five, six or seven of the substances mentioned takes place. In particular, no other addition of L-tryptophan, serotonin, long-chain neutral amino acids (LNAA) and/or alpha-lact-albumin in any combination is made. Preferably, the quotient of L-tryptophan and long-chain neutral amino acids (LNAA) does not have to be adjusted at all or only by removing L-tryptophan or long-chain neutral amino acids (LNAA).

In one embodiment of the method, which may be combined with any of the embodiments mentioned and yet to be mentioned, unless in contradiction, any melatonin is derived from the starting material. In particular, no melatonin is otherwise added.

In one embodiment of the method, which may be combined with any of the embodiments mentioned and to be mentioned, unless in contradiction, the melatonin content is adjusted by removing melatonin originating from the starting material, in particular the melatonin content is adjusted in this way such that it is at least 10 pg/mL, in particular at least 20 pg/mL, further in particular at least 30 pg/mL, and at most 60 pg/mL in the ready-to-drink infant formula.

In one embodiment of the method, which may be combined with any of the embodiments mentioned and to be mentioned, unless in contradiction, further melatonin is added to the melatonin originating from the starting material. However, the amount of further melatonin is small in relation to the amount of melatonin originating from the starting material. In this context, small means at most the same amount, in particular half as much, further in particular a quarter as much. So if the concentration of melatonin from the starting material in the finished ready-to-drink infant formula were 30 pg/mL, the further 30 pg/mL that would be necessary to achieve a final concentration of 60 pg/mL could be added otherwise in the sense of a small amount. The amount added in this example would be the same as the amount deriving from the starting material.

In one embodiment of the method, which may be combined with any of the embodiments mentioned and yet to be mentioned, unless in contradiction, at least one to all of the substances from the group of fats, triglycerides, cholesterol, iron, cortisol and cortisone originate from the starting material. This means that of the six substances mentioned, one, two, three, four, five or six of the substances get into the infant formula (exclusively) via the starting material. In particular, there is no other addition of one, two, three, four, five or six of the substances mentioned. In particular, there is no other addition of cholesterol, cortisol and/or cortisone in any combination.

One aspect of the invention relates to an infant formula, in particular an infant starter formula, according to embodiment P).

One aspect of the invention relates to an infant formula, in particular an infant starter formula, according to claim 11.

Said infant formula is characterized in that it has a melatonin content of less than 10 pg/mL in the ready-to-drink state. The preferred concentration even ranges from including 0 pg/mL up to and including 5 pg/mL or even up to and including 3 pg/mL. The low concentrations refer to an industrial infant formula suitable for daytime feeding. Alternatively, for industrial infant formula suitable for evening and night feeding, the melatonin concentration ranges from including 10 pg/mL to 60 pg/mL. Particularly preferably, it ranges from including 20 pg/mL to 40 pg/mL or even up to 50 pg/mL. In particular, it is greater than 20 pg/mL or even greater than 30 pg/mL.

While the melatonin concentration in human mature breast milk (corresponds to breast milk from the 28th day of life) is even below the detection limit during the day, measured in the early afternoon, it can be up to 99±34 pmol/L in the middle of the night in the first 3-4 days of life, but normally rather at a maximum of 40±10 pg/mL or even more specifically at 40±5 pg/mL. Thus, an industrial infant formula, which in its prepared form has a maximum concentration of melatonin such as the maximum concentration found in human breast milk, is particularly suitable for night feeding. In order to be suitable for feeding in the evening and throughout the night, the industrial infant formula should preferably have a concentration that corresponds to the average value of human breast milk over the evening and night period, i.e. at least approx. 4 pg/mL, better still approx. 10 pg/mL to 50 pg/mL or a maximum of approx. 20 pg/mL to 60 pg/mL.

An infant formula that is in powder form and is to be mixed with water has a correspondingly higher concentration of melatonin. For example, 13 g of powdered formula is mixed with 90 mL of water to obtain 100 mL of ready-to-drink formula. The melatonin concentration in the powder is therefore approx. 8 times higher than in the prepared infant formula. Said powdered infant formula is characterized by the fact that it has a melatonin content of less than 80 pg/g. The preferred concentration even ranges from including 0 pg/g to 40 pg/g. The low concentrations refer to an infant formula suitable for daytime feeding. Alternatively, in the powdered infant formula suitable for evening and night feeding, the melatonin concentration is from 80 to 480 pg/g inclusive, in particular from 150 pg/g to 400 pg/g. Particularly preferably, it ranges from 150 pg/g to 300 pg/g inclusive.

Depending on body weight, an infant drinks on average between 600 and 800 mL per day in the first 6 months of life (excluding the first days after birth). However, the range is as high as 500 to 1350 mL per day. Especially in the first weeks, the infant consumes evenly distributed over 24 h. Only when it is older, a larger proportion of the food is consumed during the day, and at night less or no food is needed. So if 600 mL are drunk over 24 h, 200 to 300 mL are consumed in the evening and at night. With a melatonin concentration of, for example, 40 pg/mL, a total of 8 or 12 ng of melatonin is ingested, for example distributed over 2, 3, 4 or even up to 6 individual meals. Accordingly, the dose of melatonin per meal is 4 ng, 2.7 ng, 2 ng or even 1.3 ng or 6 ng, 4 ng, 3 ng or even 2 ng. Contrary to conventional wisdom, it is therefore not necessary to administer a dose of 0.1 to 10 mg or even more in order to achieve an effect. Rather, the administration of melatonin at a dose of less than 0.1 mg is sufficient or even less than 10 μg and even less than 1 μg, so that a dose in this order of magnitude can be aimed for. Preferably, the dose per administration of melatonin-rich night milk as infant formula is between approx. 0.1 ng to 100 ng, in particular between 0.5 ng to 50 ng, further in particular between 1 ng or 2 ng to 20 ng. The said dose should be given once in the evening and possibly once more or even once more in the night.

In one embodiment of the infant formula, which may be combined with any of the embodiments mentioned and yet to be mentioned, unless in contradiction, the concentration of L-tryptophan in the ready-to-drink state is 25 to 37 mg/100 kcal, in particular 30 to 34 mg/100 kcal, further in particular 32 mg/100 kcal.

In one embodiment of the infant formula, which may be combined with any of the embodiments mentioned and to be mentioned, unless in contradiction, the ratio of L-tryptophan to long-chain neutral amino acids (LNAA) is 0.126 to 0.134, in particular 0.128 to 0.132, further in particular 0.130.

In one embodiment of the infant formula, which may be combined with any of the embodiments mentioned and to be mentioned, unless in contradiction, the amount of alpha-lact-albumin is 30 to 42%, in particular 33 to 39%, further in particular 36%, of the total whey protein.

A further aspect of the invention relates to a use of non-pooled milk according to claim 12.

Said use relates to the use of non-pooled milk of one mammal or several mammals, in particular cow's milk, sheep's milk, mare's milk and/or goat's milk, for the production of infant (starter) formula. In particular, the non-pooled milk is used for the production of infant (starter) formula as chrononutrition. Preferably for the production of a morning chrononutrition to wake up and/or for the production of an evening sleep-supporting chrononutrition. Chrononutrition is food that is adapted to naturally occurring chronobiological rhythms of the concentration of ingredients and to human chronobiological rhythms. The intake of this food is based on chronobiological rhythms. In the above-mentioned embodiments, this is done, for example, in accordance with day-night rhythms, so that, for example, food containing melatonin is only or preferably consumed in the evening some time before falling asleep. In the context of the present invention, the term in relation to infant nutrition is derived from the changing composition of breast milk within 24 hours, particularly in the day-night rhythm. This change affects hormones such as for example melatonin and cortisol, but also immunological parameters. Melatonin-rich infant starter formula is a particularly effective chrononutrition for infants, as they have a much longer elimination half-life of melatonin compared to adults, and they also show a relatively high maximum concentration of melatonin in the plasma after oral intake. The long half-life is probably due to the yet immature liver enzymes and yet immature kidney function. The liver enzymes are not mature before the 7th-8th month of life.

If non-pooled milk is used instead of pooled milk for the production of infant formula, this infant formula can reflect the natural change in the melatonin concentration in breast milk. Particularly suitable for this use is, for example, milk obtained as described in the methods for the production of infant formula described above. In other words, milk obtained by milking at a specific time or during a specific time range, with a specific interval between the milking processes and/or with a specific concentration of melatonin.

In one embodiment of the use, which may be combined with any of the embodiments mentioned and to be mentioned, unless in contradiction, non-pooled milk is used for the production of infant formula, in particular infant starter formula, which is obtained by milking between 3 o'clock at night and 7 o'clock in the morning. In particular, the non-pooled milk used is obtained by milking between 4 o'clock at night and 6 o'clock in the morning and preferably by milking at 5 o'clock at night±15 min. The non-pooled milk obtained as just described is used in particular for the production of an evening sleep-supporting chrononutrition.

By using non-pooled milk obtained at night or in the early morning hours, its increased melatonin concentration can be utilized, especially to obtain an infant (starter) formula that supports sleep.

In one embodiment of the use, which may be combined with any of the embodiments mentioned and to be mentioned, unless in contradiction, non-pooled milk is used for the production of infant formula, in particular infant starter formula, which is obtained by milking between 14:30 o'clock and 18:30 o'clock. In particular, the non-pooled milk used is obtained by milking between 15:30 o'clock and 17:30 o'clock and preferably by milking at 16:30 o'clock±15 min. The non-pooled milk obtained as just described is used in particular to produce a morning chrononutrition to wake up.

By using non-pooled milk obtained during the day, or in particular during the afternoon, its low melatonin concentration can be utilized, in particular to obtain an infant formula that acts as a stimulant.

In one embodiment of the use, which may be combined with any of the embodiments mentioned and yet to be mentioned, unless in contradiction, the infant formula is an infant starter formula for infants in the first six months of life or preferably even an infant starter formula for infants in the first four or even three months of life.

In principle, non-pooled milk can be used for the production of infant formula for any infant, but it is particularly preferable for the production of infant formula for younger infants, i.e. for infant starter formula, as they do not yet have a mature day-night rhythm of their own and are not able to produce a sufficient melatonin amount themselves. If they are not given any “day food” and “night food”, but simply the “24-hour food” based on pooled milk, the infants have problems finding the correct day-night rhythm or only develop it with a time delay as soon as they are able to produce sufficient melatonin themselves.

Yet another aspect of the invention relates to a medical indication according to embodiment W).

Melatonin is used in a method for the prevention and/or treatment of sleep disorders in infants, in particular in infants up to and including 4 months of age. A reduction of the melatonin level in the blood causes sleep disorders or disorders of the sleep-wake rhythm. In the first months of life, infants are unable to produce sufficient melatonin amount themselves, so it is normally supplied via breast milk. If this is not possible, the administration of melatonin can cure sleep disorders and help to regulate the sleep-wake rhythm.

Furthermore, melatonin can be used in a method for the prevention of conditions and their consequences that can arise due to melatonin deficiency in the first months of life. The dose is preferably less than 0.1 mg melatonin, even less than 10 μg and even less than 1 μg, in particular between approx. 0.1 ng to 100 ng, in particular between 0.5 ng to 50 ng, further in particular between 1 ng or 2 ng to 20 ng. It should be administered once in the evening and possibly once more or even once more at night. Furthermore, melatonin can also be used in a method to sedate infants for medical examinations such as for example magnetic resonance imaging (MRI), brainstem audiometry (BERA) and electroencephalography (EEG). These examinations are only carried out if they are absolutely necessary, as they are very stressful for the infant. In contrast to an adult, it is difficult to explain to an infant what is happening and thus create reassurance. To avoid having to give anesthetics or other strong sedatives, melatonin can be used to achieve a sedative effect. In addition, melatonin can be used in a method to support the induction of anesthesia or to reduce the after-effects of anesthesia. By taking advantage of the sedative effect, for example, less anesthetic may be necessary or the infant can be transferred to anesthesia more gently, which in turn ensures less stress. Another application of melatonin may be in a method for preventing sudden infant death syndrome (SIDS). Studies have shown that SIDS victims had lower melatonin concentrations in various body fluids compared to infants who died from other causes. Similarly, in a case-control study of infants with acute life-threatening events (ALTE), reduced concentrations of melatonin degradation products were found in urine. Furthermore, melatonin may also be used in a method for treating infants with perinatal asphyxia or hypoxic encephalopathy.

The degradation and excretion of melatonin can occur at different individual rates and, in particular, may not yet be fully developed in infants. Therefore, only relatively low dosages within the range described in the present invention should be used. In order to avoid cumulation due to insufficient excretion, at least one urine control with determination of the melatonin metabolite melatonin sulfate (synonyms: 6-hydroxymelatonin sulfate, 6-sulfatoxymelatonin) should be carried out for longer-term use (>1 week) in order to avoid overdoses. Corresponding laboratory tests are available, see e.g.: https://www.ibl-international.com/en/6-hydroxymelatonin-sulphate-melatoninsulfat-elisa.

In one embodiment of the medical indication, which may be combined with any of the embodiments mentioned and yet to be mentioned, unless in contradiction, melatonin is used in a method for treating sleep disorders in infants, in particular in infants aged up to 4 months, wherein the melatonin is given in the evening, and if necessary additionally at night, preferably with or in the infant formula, in particular the infant starter formula. Melatonin can also be used in this way in a method for preventing conditions and their consequences that can arise due to melatonin deficiency in the first months of life. The dose is preferably less than 0.1 mg melatonin, even less than 10 μg and even less than 1 μg, in particular between approx. 0.1 ng to 100 ng, in particular between 0.5 ng to 50 ng, further in particular between 1 ng or 2 ng to 20 ng. It should be given once in the evening and possibly once more or even once more at night.

As already described, melatonin essentially regulates the sleep-wake-rhythm, so that infants who have not yet developed it sufficiently and therefore have sleep disorders can be helped to avoid and/or treat the sleep disorder by administering melatonin in the evening and/or at night. Evening administration is particularly preferred as melatonin is best effective in the evening and at night because the associated melatonin receptors are particularly sensitive to melatonin in the evening and at night and are less active during the day (daytime light-dependent activity). Before administering melatonin as an externally administered drug (i.e. as single substance), an attempt can or should even be made to administer infant formula based on (predominantly) non-pooled milk as starting material. With regard to chronobiological and sleep medicine effects, melatonin only appears to be effective in the sensitive periods from 20:00 o'clock in the evening to 03:00 o'clock in the morning—due to the underlying phase response curve (PRC), which in turn is shaped by so-called Timer and clock genes and is influenced or modified to a limited extent by other factors. Taking melatonin during the day therefore has hardly any effect.

Yet another aspect of the invention relates to a medical indication according to claim 13.

Specifically, an infant formula, in particular an infant starter formula, containing melatonin is used in a method for treating sleep disorders in infants, in particular in infants up to and including 4 months of age. Such an infant (starter) formula may further be used in a method for the prevention of conditions and their consequences which may result from melatonin deficiency in the first months of life. The dose is preferably less than 0.1 mg melatonin, even less than 10 μg and even less than 1 μg, in particular between approx. 0.1 ng to 100 ng, in particular between 0.5 ng to 50 ng, further in particular between 1 ng or 2 ng to 20 ng. It should be given once in the evening and possibly once more or even once more at night.

In principle, one tries to avoid giving infants any form of medication, so that the melatonin is optimally supplied with an infant formula that preferably naturally has a suitable melatonin concentration (e.g. 20-50 pg/mL; preferred concentrations can be found, for example, in the description of the method and the use) due to the choice of milk, which forms the starting material of the infant (starter) formula. This solves on one hand the problem of how to administer the melatonin to the infant as a drug, and on the other hand any intolerance to an excipient or the like cannot happen.

Yet another aspect of the invention relates to a medical indication formulated in the Swiss-type claim formulation according to embodiment Y).

This relates to the use of melatonin for the production of a medicament for the treatment of sleep disorders in infants up to and including 4 months of age. Further, melatonin may be used for the production of a medicament for the prevention of conditions and their consequences that may arise from melatonin deficiency in the first months of life. As can be seen from the previous embodiments, the “medicine” can also be an infant (starter) formula having a suitable melatonin concentration (e.g. 20-50 pg/mL). The dose is preferably less than 0.1 mg melatonin, even less than 10 μg and even less than 1 μg, in particular between approx. 0.1 ng to 100 ng, in particular between 0.5 ng to 50 ng, further in particular between 1 ng or 2 ng to 20 ng. It should be given once in the evening and possibly once more or even once more at night.

In one embodiment of melatonin for the use in a method for treating or preventing a disease or the like, or a use of melatonin which may be combined with any of the embodiments mentioned and yet to be mentioned, unless in contradiction, the melatonin is provided as an aqueous solution in a liquid, such as for example water, juice or milk. Alternatively, the melatonin is provided as a spray with nanoparticles and/or with liposomes.

Since melatonin is stable in aqueous solution and among others also in dissolved form in orange juice, milk and similar forms of preparation with an ambient pH<7.4, administration in liquid form in time conjunction with the administration of infant starter formula and/or with breastfeeding or partial breastfeeding+infant starter formula does not pose a problem.

In one embodiment of melatonin for the use in a method for treating or preventing a disease or the like, or a use of melatonin which may be combined with any of the above and yet to be mentioned embodiments unless in contradiction, the melatonin is not provided as a retard preparation.

Since melatonin triggers pulsatile chronobiological effects, retard preparations should not be used in infancy.

In one embodiment of melatonin for the use in a method for treating or preventing a disease or the like or a use of melatonin or a melatonin-containing infant formula, which may be combined with any of the embodiments mentioned and yet to be mentioned, unless in contradiction, the amount of melatonin per dose is between 0.1 to 100 ng, in particular between 0.5 ng to 50 ng, further in particular between 1 ng or 2 ng to 20 ng. It should be administered, for example, once and possibly once more or even once more per day or application (e.g. sedation prior to examination). It goes without saying that the declared dose is precise and may only be subject to a slight fluctuation, such as e.g. of 2 or 5%. This is not the case with food supplements; the declared doses fluctuate in some cases between +465 to −80% compared to the concentrations that can actually be detected.

In one embodiment of melatonin for the use in a method for treating or preventing a disease or the like, or a use of melatonin which may be combined with any of the embodiments mentioned and to be mentioned, unless in contradiction, the melatonin is applied nasally, in particular by means of a nasal spray. Preferably, the melatonin is provided in the form of encapsulated nanoparticles. The concentration of melatonin is selected in particular such that the dose is preferably below 0.1 mg melatonin, even below 10 μg and even below 1 μg, in particular between approx. 0.1 ng to 100 ng, in particular between 0.5 ng to 50 ng, further in particular between 1 ng or 2 ng to 20 ng. The dose is applied, for example, via one or two spray shots with the nasal spray. For example, one spray shot has a volume of 0.05 to 0.15 mL, in particular 0.08-1.0 mL, further in particular 0.09 mL.

In one embodiment of melatonin for the use in a method for treating or preventing a disease or the like, or a use of melatonin which may be combined with any of the embodiments mentioned and to be mentioned, unless in contradiction, the melatonin is administered sublingually, in particular by means of a spray, melting tablets or chewable capsules. Melatonin can also be provided here for sublingual application in the form of capsulated nanoparticles. The concentration of melatonin is selected in particular so that the dose is preferably below 0.1 mg melatonin, even below 10 μg and even below 1 μg, in particular between approx. 0.1 ng to 100 ng, in particular between 0.5 ng to 50 ng, further in particular between 1 ng or 2 ng to 20 ng. The dose is applied, for example, via one or two spray shots with the spray. For example, one spray shot has a volume of 0.05 to 0.15 mL, in particular of 0.08-1.0 mL, further in particular of 0.09 mL.

Yet another aspect of the invention relates to a method of adding melatonin according to claim 15.

The addition of melatonin is thereby made to an infant formula, in particular to an infant starter formula prepared starting from pooled milk of one or more mammals or starting from non-pooled milk of one or more mammals. The non-pooled milk is, for example, an infant formula prepared according to a method as described in any one of the claims 1 to 8 or any one of the embodiments A) to H).

In one embodiment of the method for adding melatonin, which may be combined with any of the embodiments mentioned and yet to be mentioned, unless in contradiction, the concentration of melatonin is adjusted such that it is at least 10 pg/mL, in particular 20 pg/mL, further in particular 30 pg/mL, and the maximum value is 60 pg/mL in the ready-to-drink infant formula.

The invention is explained in more detail below using examples.

### Example 1

It is mid-June (i.e. summertime) and a Swiss farmer provides two types of milk for the production of infant (starter) formula for a food processing company. The first type of milk is the milk of several cows that he has milked on his farm between 4:00 and 5:00 o'clock in the morning, the second type of milk is also the milk of several cows, but he has milked it on his farm between 20:00 and 21:00 o'clock. Both types of milk are non-pooled milk, as the milks obtained at different specific times of the day are not mixed together, so that the chronobiological change in melatonin concentration within the day is maintained by separating the first and second type of milk. The first type, which is melatonin-rich (contains, for example, 80% of the melatonin produced by the cows in a day), is used to produce a sleep-supporting chrononutrition, while the second type of milk, which is melatonin-poor (contains, for example, 20% of the melatonin produced by the cows in a day), is used to produce a stimulating chrononutrition.

### Example 2

A treatment is to be offered for infants with problems falling asleep. Milk from one or more cows is obtained by milking in the middle of the night, e.g. one or two hours before sunrise. The milking process carried out immediately before the night milking in turn took place one to two hours after sunset in order to remove the melatonin-poor milk produced during the day from the udder. The milk obtained by night milking has an especially high melatonin content (e.g. 30 pg/mL to 40 pg/mL) and the infant (starter) formula produced from it can be administered, for example, to treat (falling) sleep disorders in the infant.

### Example 3

A non-pooled milk is provided, obtained by milking goats during a specific time window of the day of approximately one hour, e.g. between 16:00 and 17:00 o'clock. The previous milking process already took place at sunrise. Said non-pooled goat's milk contains a melatonin content of less than 10 pg/mL and is used for the production of infant (starter) formula, which is intended as chrononutrition to wake up the infant and is therefore fed to the infant during the day.

### Example 4

Melatonin, in particular obtained from non-pooled milk (e.g. cow's or sheep's milk) at times when it is dark; (the previous milking process still took place at a time when it was light), is administered to the infant in the evening to prevent sleep disorders. It can be administered via an infant (starter) formula produced from the non-pooled infant milk, which contains at least 20 pg/mL melatonin in the ready-to-drink form.

### Example 5

It is mid-November (i.e. winter time) and a Swiss farmer provides two types of milk for the production of infant (starter) formula for a food processing company. The first type of milk is the milk of several cows, which are milked within a time window of 6 h. Some are milked on his farm between 00:00 and 1:00 o'clock in the morning, others between 2:00 and 3:00 o'clock in the morning and others between 5:00 and 6:00 o'clock in the morning. The time before, the cows were milked at sunset, i.e. around 16:45 o'clock. The second type of milk is also the milk of several cows, which are milked within a time window of 6 h. Some are milked on the farm between 12:00 and 13:00 o'clock at midday, others between 14:00 and 15:00 o'clock in the afternoon and the others between 17:00 and 18:00 o'clock. The time before, these cows were each milked at sunrise, i.e. around 7:30 o'clock. Both types of milk are non-pooled milk in the sense of the invention, since the milks obtained at different time windows are not mixed with each other, so that the chronobiological change in the melatonin concentration within the day is maintained via the separation of the first and second type of milk. The first type, which is melatonin-rich (contains, for example, 85% of the melatonin produced by the cows in one day and/or a concentration of 30 pg/mL), is used to produce a sleep-supporting chrononutrition, the second type of milk, which is melatonin-poor (contains, for example, 15% of the melatonin produced by the cows in one day and/or a concentration of melatonin of 5 pg/mL), is used to produce a stimulating chrononutrition.

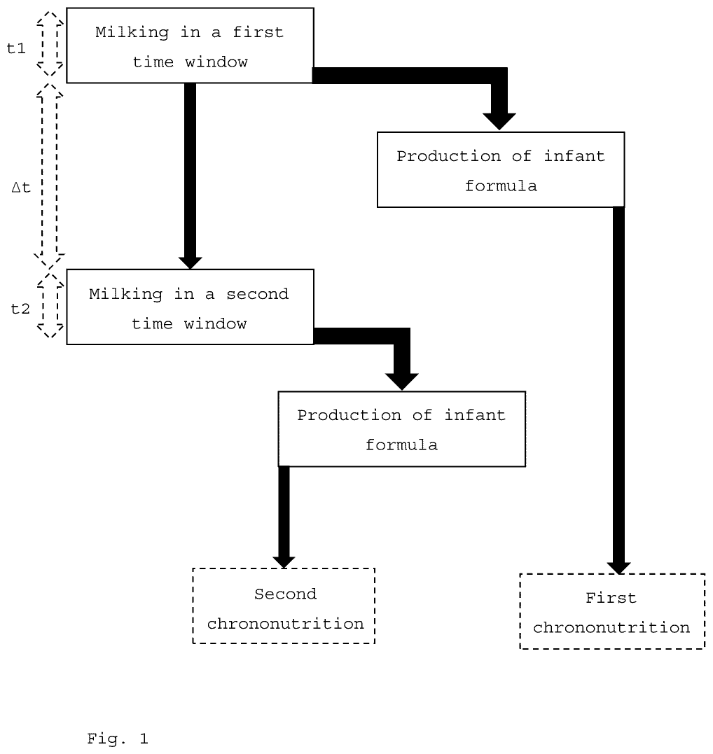

An embodiment of the present invention is explained in more detail below with reference to a FIGURE. FIG. 1 shows an embodiment of the two-part method.

FIG. 1 shows an embodiment of the two-part method. It can be seen how a first milking of one or more mammals of the same species, such as for example a cow, sheep, goat or horse, is milked in a specific time window t1. The time window is, for example, one hour long, starts at 4:30 o'clock and ends at 5:30 o'clock, shortly before sunrise. Melatonin-rich “night milk” is collected, which is then further processed into infant (starter) formula, which is suitable for the evening and night feeding and can optionally be distributed as a first chrononutrition for promoting falling asleep. After a period of time Δt of, for example, 13 h, milking then takes place again in the time window t2, which is, for example, one and a half hours long, starts at 19:30 o'clock and ends at 21:00 o'clock, shortly before sunset. Melatonin-poor “day milk” is collected, which is then further processed into infant (starter) formula that is suitable for feeding during the day and can optionally be distributed as a second chrononutrition for waking up.

For maintaining the originally filed disclosure, further embodiments of the invention are mentioned below as embodiments A) to Z10):

A) Method according to any one of the claims 1 to 3, wherein the milk provided as starting material is obtained by milking between 14:30 o'clock and 18:30 o'clock, in particular between 15:30 o'clock and 17:30 o'clock, preferably at 16:30 o'clock±15 min.

B) Method according to any one of the claims 1 to 3 or according to embodiment A), wherein the milk used as starting material has a melatonin content of below 10 pg/mL, in particular of below 8 pg/mL, further in particular of below 5 pg/mL, preferably from including 0 pg/mL up to and including 3 pg/mL.

C) Method according to any one of the claims 1 to 5, or according to embodiment A) or B), wherein the non-pooled milk is obtained by a milking process carried out between 6 hours and 18 hours, in particular between 9 hours and 15 hours, preferably between 11 hours and 13 hours, after the previous milking process.

D) Method according to any one of the claims 1 to 5 or embodiment C), wherein the milk used as starting material is obtained by a milking process carried out shortly before or at sunrise.

E) Method according to any one of the claims 1 to 3 or any one of the embodiments A) to C), wherein the milk used as starting material is obtained by a milking process carried out shortly before or at sunset.

F) Method according to any one of the embodiments A) to E), wherein the L-tryptophan concentration, in particular of the starting material or the first starting material, is adjusted, preferably in such a way that in the ready-to-drink infant starter formula it amounts to a concentration from 25 to 37 mg/100 kcal, in particular from 30 to 34 mg/100 kcal, further in particular of 32 mg/100 kcal.

G) Method according to any one of the embodiments A) to E), wherein the quotient of L-tryptophan to long-chain neutral amino acids (LNAA) is adjusted, preferably such that it is 0.126 to 0.134, in particular 0.128 to 0.132, further in particular 0.130, in the ready-to-drink infant formula.

H) Method according to any one of the claims 1 to 8 or any one of the embodiments A) to G), wherein alpha-lact-albumin is added, preferably in such a way that it amounts to 30 to 42%, in particular 33 to 39%, further in particular 36%, of the whey protein.

I) Method according to any one of the claims 1 to 8, or any one of the embodiments A) to H), wherein the one mammal or the several mammals, when the animals are kept in the stable, are exposed to a bright light during the day of at least 200 lux for at least 12 h, in particular for at least 14 h, further in particular for at least 16 h, which light preferably has a high proportion of blue light and, for example, has a wavelength of 465 nm, and wherein the one mammal or the several mammals, when the animals are kept in the stable at night, are exposed to darkness for at least 8 h, in particular for at least 10 h, further in particular for at least 12 h.

J) Method according to any one of the claims 1 to 8, or any one of the embodiments A) to I), wherein the non-pooled milk is not heated above 65° C., in particular not above 62° C., further in particular not above 60° C., during the method and/or the non-pooled milk is exposed to a pH value of at most 7.4 during the entire method.

K) Method according to any one of the embodiments A) to J), wherein at least one to all of the substances from the group of L-tryptophan, 5-HTP, serotonin, acetyl-serotonin, melatonin, long-chain neutral amino acids (LNAA) and alpha-lact-albumin originate from the starting material and, in particular, at least one to all of the substances from the said group are not added otherwise.

L) Method according to any one of the embodiments A) to J), wherein any melatonin is derived from the starting material and in particular no melatonin is otherwise added.

M) Method according to claim 10 or embodiment L), wherein the melatonin content is adjusted by removing melatonin originating from the starting material, in particular the melatonin content is adjusted in this way such that it is at least 10 pg/mL, in particular at least 20 pg/mL, further in particular at least 30 pg/mL, and at most 60 pg/mL in the ready-to-drink infant formula.

N) Method according to any one of the claims 1 to 8, or any one of the embodiments A) to J), wherein further melatonin is added to the melatonin originating from the starting material, wherein the amount of further melatonin is small in relation to the amount of melatonin originating from the starting material, wherein small means at most the same, in particular half as much, further in particular a quarter as much.

O) Method according to any one of the claims 1 to 10, or any one of the embodiments A) to N), wherein at least one to all of the substances from the group of fats, triglycerides, cholesterol, iron, cortisol and cortisone originate from the starting material and, in particular, at least one to all of the substances from the group are not added otherwise.

P) Industrial infant formula suitable for humans aged from 0 to 12 months, in particular industrial infant starter formula suitable for humans aged from 0 to 6 months, produced by a method according to any one of the claims 1 to 10, or any one of the embodiments A) to O).

Q) Infant formula according to claim 11 or embodiment P), wherein it has a concentration of 25 to 37 mg/100 kcal, in particular 30 to 34 mg/100 kcal, in particular 32 mg/100 kcal, of L-tryptophan in the ready-to-drink state.

R) Infant formula according to claim 11 or embodiment P) or Q), wherein in the ready-to-drink state it has a ratio of L-tryptophan to long-chain neutral amino acids (LNAA) of from 0.126 to 0.134, in particular from 0.128 to 0.132, further in particular of 0.130.

S) Infant formula according to claim 11 or any one of the embodiments P) to R), wherein alpha-lact-albumin makes up 30 to 42%, in particular 33 to 39%, further in particular 36%, of the whey protein.

T) Use according to claim 12, wherein the non-pooled milk is obtained by milking between 3 o'clock at night and 7 o'clock in the morning, in particular between 4 o'clock at night and 6 o'clock in the morning, further in particular at 5 o'clock at night±15 min, preferably at sunrise, for the production of infant formula, in particular as evening sleep-supporting chrononutrition.

U) Use according to claim 12, wherein the non-pooled milk is obtained by milking between 14:30 o'clock and 18:30 o'clock, in particular between 15:30 o'clock and 17:30 o'clock, further in particular at 16:30 o'clock±15 min, preferably at sunset, for the production of infant formula, in particular as morning chrononutrition to wake up.

V) Use according to claim 12 or embodiment T) or U), wherein the industrial infant formula is an infant starter formula in particular for infants in the first six months of life, preferably in the first four or even three months of life.

W) Melatonin for use in a method for:
preventing and/or treating sleep disorders in infants, in particular in infants up to and including 4 months of age; and/or
sedation of infants for medical examinations such as for example magnetic resonance tomography (MRI), brainstem audiometry (BERA) and electroencephalography (EEG); and/or
supporting the induction of anesthesia and/or reducing after-effects due to anesthesia;
prevention of sudden infant death (SIDS); and/or
treatment of infants with perinatal asphyxia; and/or
treatment of infants with hypoxic encephalopathy.

X) Melatonin for use in a method for preventing and/or treating sleep disorders in infants, in particular in infants up to and including 4 months of age, wherein the melatonin is administered in the evening, and if necessary additionally at night, preferably with or in the infant formula.

Y) Use of melatonin for the production of a medicament for:
preventing and/or treating sleep disorders in infants, in particular in infants up to and including 4 months of age; and/or
sedation of infants for medical examinations such as magnetic resonance tomography (MRI), brainstem audiometry (BERA) and electroencephalography (EEG); and/or
supporting the induction of anesthesia and/or reducing after-effects due to anesthesia;
prevention of sudden infant death (SIDS); and/or
treatment of infants with perinatal asphyxia; and/or
treatment of infants with hypoxic encephalopathy.

Z1) Melatonin for use in a method for the prevention of conditions and their consequences which may arise from melatonin deficiency in the first months of life, in particular in infants up to and including 4 months of age.

Z2) Melatonin for use in a method for the prevention of conditions and their consequences which may arise from melatonin deficiency in the first months of life, wherein the melatonin is administered in the evening, and if necessary additionally at night, preferably with or in the infant formula.

Z3) Use of melatonin for the production of a medicament for the prevention of conditions and their consequences which may arise from melatonin deficiency in the first months of life, in particular in infants up to and including 4 months of age.

Z4) Melatonin according to any one of the embodiments W), X), Z1), and Z2) or a use of melatonin according to embodiment Y) or Z3), wherein the melatonin is provided as an aqueous solution in a liquid, in particular water, juice or milk, or as spray with nanoparticles and/or with liposomes.

Z5) Melatonin according to any one of the embodiments W), X), Z1), and Z2) or a use of melatonin according to embodiment Y) or Z3), wherein the melatonin is not provided as a retard preparation.

Z6) Melatonin according to any one of the embodiments W), X), Z1), and Z2), a melatonin-containing industrial infant formula according to claim 13 or 14, or a use of melatonin according to embodiment Y) or Z3), wherein the amount of melatonin per dose is between 0.1 to 100 ng, in particular between 0.5 ng to 50 ng, further in particular between 1 ng or 2 ng to 20 ng.

Z7) Melatonin according to any one of the embodiments W), X), Z1), Z2), Z4) and Z5), wherein the melatonin is provided nasally, in particular by means of a nasal spray, further in particular as encapsulated nanoparticles.

Z8) Melatonin according to any one of the embodiments W), X), Z1), Z2), Z4) and Z5), wherein the melatonin is provided sublingually, in particular by means of a spray, melting tablets or chewable capsules, further in particular as encapsulated nanoparticles.

Z9) Method comprising:
the addition of melatonin to industrial infant formula suitable for humans aged 0 to 12 months, in particular industrial infant starter formula suitable for humans aged 0 to 6 months, prepared starting from pooled milk of one or several mammals; or
the addition of melatonin to industrial infant formula suitable for humans aged from 0 to 12 months, in particular infant starter formula suitable for humans aged from 0 to 6 months, prepared starting from non-pooled milk of one or several mammals, in particular to an infant formula prepared according to a method according to any one of the embodiments A) to O).

Z10) Method according to claim 15 or according to embodiment Z9), wherein the concentration of melatonin is adjusted such that it is at least 10 pg/mL, in particular 20 pg/mL, further in particular 30 pg/mL, in the ready-to-drink infant formula, and the maximum value is 60 pg/mL.

1. Method for producing industrial infant formula suitable for humans aged from 0 to 12 months, in particular industrial infant starter formula suitable for humans aged from 0 to 6 months, characterized in that non-pooled milk of one or several animals of the genus mammal, in particular of the genus even-toed ungulates, further in particular cow's milk, sheep's milk, mare's milk, donkey's milk and/or goat's milk, is provided as starting material,
wherein non-pooled milk is to be understood as milk from a single milking process or milk from several milking processes, wherein the single or the several milking processes are carried out at a specific time or in a specific time window in relation to a 24-hour cycle, wherein the specific time window is less than 6 h, in particular less than 3 h, further in particular less than 2 h.

2. Method according to claim 1, wherein, in relation to the total quantity of milk as starting material, the proportion of non-pooled milk is at least 50%ᵥₒₗ, in particular at least 75%ᵥₒₗ, further in particular at least 90%ᵥₒₗ.

3. Method according to claim 1, wherein exclusively non-pooled milk is provided as starting material.

4. Method according to claim 1, wherein the milk provided as starting material is obtained by milking between 3 o'clock at night and 7 o'clock in the morning, in particular between 4 o'clock at night and 6 o'clock in the morning, preferably at 5 o'clock at night±15 min.

5. Method according to claim 1, wherein the milk provided as starting material has a content of melatonin of including 10 pg/mL to 60 pg/mL, in particular of including 20 pg/mL to 50 pg/mL, preferably of including 20 pg/mL to 40 pg/mL.

6. Method according to claim 1, characterized in that the method is a two-part method, wherein:
in the first part, melatonin-rich milk is obtained as the first starting material by milking at a first time or in a first time window;
in the second part, melatonin-poor milk is obtained as a second starting material by milking at a second time or in a second time window;
the first time or the first time window is between 3 o'clock at night and 7 o'clock in the morning, in particular between 4 o'clock at night and 6 o'clock in the morning, preferably at 5 o'clock at night±15 min, or at sunrise;
the second time or the second time window is between 14:30 o'clock and 18:30 o'clock, in particular between 15:30 o'clock and 17:30 o'clock, preferably at 16:30 o'clock #15 min, or at sunset; and
the interval between the first and the second part of the method is between 6 hours and 18 hours, in particular between 9 hours and 15 hours, preferably between 11 hours and 13 hours; and wherein the first starting material and the second starting material are processed separately, namely into a first melatonin-rich infant formula and a second melatonin-poor infant formula.

7. Method according to claim 1, wherein the L-tryptophan concentration, in particular of the starting material or of the first starting material, is adjusted, preferably such that it is a concentration of 25 to 37 mg/100 kcal, in particular of 30 to 34 mg/100 kcal, further in particular of 32 mg/100 kcal, in the ready-to-drink infant starter formula.

8. Method according to claim 1, wherein the quotient of L-tryptophan to long-chain neutral amino acids (LNAA) is adjusted, preferably in such a way that it is 0.126 to 0.134, in particular 0.128 to 0.132, further in particular 0.130, in the ready-to-drink infant formula.

9. Method according to claim 1, wherein at least one to all of the substances from the group L-tryptophan, 5-HTP, serotonin, acetyl-serotonin, melatonin, long-chain neutral amino acids (LNAA) and alpha-lact-albumin originate from the starting material and in particular at least one to all of the substances from the said group are not otherwise added.

10. Method according to claim 1, wherein any melatonin originates from the starting material and in particular no melatonin is otherwise added.

11. Infant formula, in particular infant starter formula, characterized in that it has a melatonin content in ready-to-drink state of less than 10 pg/mL, preferably from 0 pg/mL to 5 pg/mL inclusive, or from 10 pg/mL to 60 pg/mL inclusive, preferably from 20 pg/mL to 40 pg/mL inclusive.

12. Use of non-pooled milk of one or several animals of the genus mammals, in particular cow's milk, sheep's milk, mare's milk, donkey's milk and/or goat's milk, for the production of industrial infant formula suitable for humans aged from 0 to 12 months, in particular for the production of industrial infant formula as chrononutrition, preferably as morning chrononutrition to wake up and/or as evening sleep-supporting chrononutrition.

13. Melatonin-containing industrial infant formula for use in a method for:
preventing and/or treating sleep disorders in infants, in particular in infants up to and including 4 months of age; and/or
sedation of infants for medical examinations such as for example magnetic resonance tomography (MRI), brainstem audiometry (BERA) and electroencephalography (EEG); and/or
supporting the induction of anesthesia and/or reducing after-effects due to anesthesia;
prevention of sudden infant death (SIDS); and/or
treatment of infants with perinatal asphyxia; and/or
treatment of infants with hypoxic encephalopathy.

14. Melatonin-containing industrial infant formula for use in a method for the prevention of conditions and their consequences which may arise from melatonin deficiency in the first months of life, in particular in infants up to and including 4 months of age.

15. Method comprising:
the addition of melatonin to industrial infant formula suitable for humans aged from 0 to 12 months, in particular industrial infant starter formula suitable for humans aged from 0 to 6 months, prepared starting from pooled milk of one or several mammals; or
the addition of melatonin to industrial infant formula suitable for humans aged from 0 to 12 months, in particular infant starter formula suitable for humans aged from 0 to 6 months, prepared from non-pooled milk of one or several mammals, in particular to an infant formula prepared by a method according to claim 1
